(19) United States
(12) Patent Application Publication
(10) Pub. No.: US 2014/0046304 A1
(43) Pub. Date: Feb. 13, 2014

(54) MEDICAL INSTRUMENT ADAPTOR AND METHODS OF USE

(71) Applicant: Boston Scientific Scimed, Inc., Maple Grove, MN (US)

(72) Inventors: Norman MAY, Northborough, MA (US); Gary KAPPEL, Acton, MA (US); Sean FLEURY, Brighton, MA (US); Mark WOOD, Shrewsbury, MA (US)

(73) Assignee: Boston Scientific Scimed, Inc., Maple Grove, MN (US)

(21) Appl. No.: 13/959,286

(22) Filed: Aug. 5, 2013

Related U.S. Application Data
(60) Provisional application No. 61/682,550, filed on Aug. 13, 2012.

(51) Int. Cl. A61B 17/00

(52) U.S. Cl. CPC A61B 17/00
USPC 606/1

(57) ABSTRACT

Embodiments of the disclosure include an adaptor for a medical instrument configured for insertion into a body of a patient. The adaptor may comprise a housing defining an internal recess configured to receive a portion of the medical instrument. The internal recess may further include geometric configurations configured to secure the housing to the medical instrument. The outer configuration of the adaptor may be compatible with a separate medical device.

### CROSS-REFERENCE TO RELATED APPLICATIONS

This application claims the benefit of priority from U.S. Provisional Application No. 61/682,550, filed on Aug. 13, 2012, the entirety of which is incorporated by reference herein.

### FIELD OF THE DISCLOSURE

Embodiments of the present disclosure relate to adaptors, and more particularly, adaptors for altering the structure or function of medical instruments.

### BACKGROUND OF THE DISCLOSURE

Minimally invasive surgical devices, such as endoscopic and laparoscopic devices, provide access to surgical sites while minimizing patient trauma. The growing capabilities of such therapeutic devices allow physicians to perform an increasing variety of surgeries through minimally invasive routes. This increasing variety of surgeries presents new challenges for device manufacturers and practitioners, because a greater variety of instruments is required.

As medical technology advances at a rapid pace, hospitals and healthcare providers may find it difficult to keep up with the changing technology. Newer models may come to market in quick succession, each offering more features or more enhanced features than the one before. Often, because of the differences in features between models, the accompanying instruments and accessories for each model may differ such that instruments for use with older or lower-end models may not be compatible with those for newer or higher-end models. Accordingly, healthcare providers may not be able to afford upgrading to the newest technology because an entirely new supply of instruments would need to be purchased.

Further, hospitals may own several different models or brands of similarly functioning devices. Maintaining an inventory of instruments for each separate model or brand can waste limited space and money, cause unnecessary inventory issues, and lead to confusion for healthcare providers who must switch between the different models. Accordingly, a need exists for an adaptor capable of making a variety of instruments compatible with a variety of different medical devices. The present disclosure aims to overcome at least some of these deficiencies in the prior art.

### SUMMARY OF THE DISCLOSURE

Embodiments of the present disclosure relate to adaptors, and more particularly, adaptors for altering the structure or function of medical instruments. Various embodiments of the disclosure may include one or more of the following aspects.

In accordance with one embodiment, an adaptor for a medical instrument may be configured for insertion into a body of a patient. The adaptor may comprise a housing defining an internal recess configured to receive a portion of the medical instrument. The internal recess may further include geometric configurations configured to secure the housing to the medical instrument. The outer configuration of the adaptor may be compatible with a medical device.

Various embodiments of the adaptor may include one or more of the following features: a housing having a plurality of sections; the outer configuration of the adaptor may differ from the outer configuration of the medical instrument in one or more of shape, size, and function; a proximal portion of the housing may be configured to receive a handle portion of the medical instrument; the medical instrument may include an elongate shaft having proximal and distal ends, a handle disposed at the proximal end of the elongate shaft, and an end-effector extending from the distal end of the elongate shaft; an internal recess with one or more supporting structures for supporting a portion of the medical instrument therein; the supporting structures may include a plurality of circumferential ribs; the housing may include a proximal portion having a first diameter and a distal portion having a second diameter different from the first diameter; a plurality of sections may be configured to be secured together by, for example, one of an interference fit, a snap fit, and a friction fit; the housing may be directly connected to the medical instrument; and one or more control elements may be configured to control one or more features of the medical instrument that the medical instrument is not capable of without the adaptor, but is capable of when the adaptor is mounted on the medical instrument.

In accordance with another embodiment, a medical system may include an introduction sheath having a proximal end, a distal end, and a lumen extending therebetween. The proximal end may include a handle having a port in communication with the lumen. The medical system may further include a medical instrument having an outer configuration incompatible with the port. The medical system may also include an adaptor having a housing defining an internal recess. The internal recess may be configured to receive a portion of the medical instrument and may generally correspond to an outer configuration of the portion of the medical instrument. The adaptor may include a securing mechanism to secure the housing about the medical instrument. The outer configuration of the adaptor may also be compatible with the port.

Various embodiments of the medical system may include one or more of the following features: the introduction sheath may include an endoscope having a plurality of lumens; the housing may include a plurality of sections; the sections may be configured to be secured together by, for example, one of an interference fit, a snap fit and a friction fit; the outer configuration of the adaptor may differ from the outer configuration of the medical instrument, for instance, in one or more of shape, size, and function; a proximal portion of the housing may be configured to receive a handle portion of the medical instrument; and the adaptor may be configured for use with more than one type of medical instrument or more than one type of introduction sheath.

In accordance with another embodiment, a medical method may include the steps of advancing an introduction sheath having a working channel to a target location within the body of a patient. An adaptor may then be secured to an exterior of a medical instrument if the exterior of the medical instrument includes a geometry that is incompatible with a geometry of the working channel. The adaptor may include a housing defining an internal recess configured to receive a portion of the medical instrument. The internal recess may be configured to generally correspond to an outer configuration of the portion of the medical instrument. The adaptor may have an outer configuration that is compatible with the working channel. Once the adaptor is secured on the medical instrument, the medical instrument may be advanced into the working channel of the introduction sheath.

Various embodiments of the medical method may also include a housing having a plurality of sections.

In this respect, before explaining multiple embodiments of the present disclosure in detail, it is to be understood that the present disclosure is not limited in its application to the details of construction and to the arrangements of the components set forth in the following description or illustrated in the drawings. The present disclosure is capable of embodiments in addition to those described and of being practiced and carried out in various ways. Also, it is to be understood that the phraseology and terminology employed herein, as well as in the abstract, are for the purpose of description and should not be regarded as limiting.

The accompanying drawings illustrate certain exemplary embodiments of the present disclosure and, together with the description, serve to explain the principles of the present disclosure. As such, those skilled in the art will appreciate that the conception upon which this disclosure is based may readily be used as a basis for designing other structures, methods, and systems for carrying out the several purposes of the present disclosure. It is important, therefore, to recognize that the claims should be regarded as including such equivalent constructions insofar as they do not depart from the spirit and scope of the present disclosure.

### BRIEF DESCRIPTION OF THE DRAWINGS

The accompanying drawings illustrate certain exemplary embodiments of the present disclosure, and together with the description, serve to explain principles of the present disclosure.

FIG. 1 depicts a perspective view of an exemplary adaptor shown mounted on an exemplary medical instrument, in accordance with an embodiment of the present disclosure;

FIG. 2 depicts an exploded view of the exemplary adaptor removed from the exemplary medical instrument of FIG. 1, in accordance with an embodiment of the present disclosure;

FIG. 3 depicts a perspective view of an exemplary modular adaptor shown mounted on an exemplary medical instrument, in accordance with an embodiment of the present disclosure; and

FIG. 4 depicts an exploded view of the exemplary modular adaptor of FIG. 3, in accordance with an embodiment of the present disclosure.

### DETAILED DESCRIPTION OF THE PREFERRED EMBODIMENTS

Reference will now be made in detail to the exemplary embodiments of the present disclosure described above and illustrated in the accompanying drawings. Wherever possible, the same reference numbers will be used throughout the drawings to refer to same or like parts.

While the present invention is described herein with reference to illustrative embodiments for particular applications, such as, for use in connection with endoscopic or laparoscopic instruments or surgical systems, it should be understood that the embodiments described herein are not limited thereto. Those having ordinary skill in the art and access to the teachings provided herein will recognize additional modifications, applications, embodiments, and substitution of equivalents all fall within the scope of the embodiments disclosed herein. For instance, the principles described herein may be used with any suitable medical device (e.g., standard surgical instruments, minimally invasive surgical instruments, forceps, biopsy needles, snares, catheters, scissors, etc.) for any suitable treatment or diagnostic purpose (e.g. surgery, biopsies, drug delivery, etc.). Further, the adaptor may be used with non-medical instruments and devices, such as, for instance, instruments for the inspection or repair of machinery. Accordingly, the disclosure is not to be considered as limited by the foregoing or following descriptions.

Other features and advantages and potential uses of the present disclosure will become apparent to someone skilled in the art from the following description of the disclosure, which refers to the accompanying drawings.

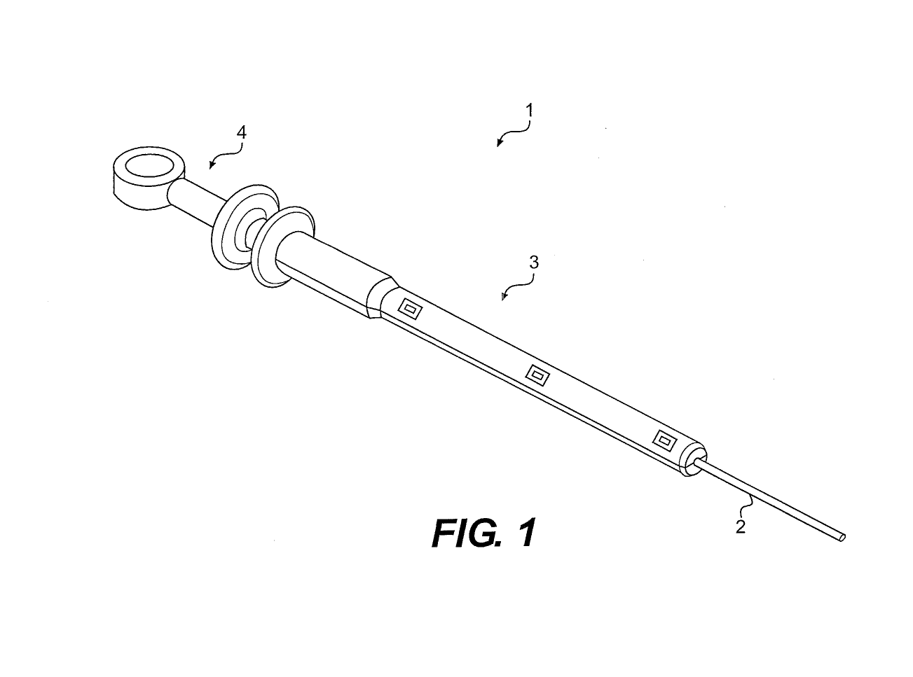

FIG. 1 illustrates an exemplary adaptor 3 of the present disclosure when mounted on an exemplary medical instrument 1. Medical instrument 1 can be configured for use with therapeutic or diagnostic procedures within a patient's body. For example, medical instrument 1 can be configured for use with an endoscope, a laparoscope, a guide tube, an access catheter, or any other suitable type of device configured to access a patient's body. During these procedures, the site of operation may be located deep within a patient's body. For instance, medical instrument 1 may be used for procedures with surgical sites located within or adjacent to various body organs, such as, an esophagus, a heart, a stomach, a pelvic area, a bladder, an intestine, or any other portion of a gastrointestinal, urinary, or pulmonary tract. Accordingly, medical instrument 1 may be advanced to an internal site with the aid of an introducer (not shown), e.g., an endoscope or other suitable introduction sheath.

The introducer may be inserted through an incision or a natural anatomical opening, and medical instrument 1 may access the patient's body via insertion through the introducer. In some embodiments, medical instrument 1 may be used in natural orifice transluminal endoscopic surgery (NOTES) procedures or single incision laparoscopic surgical (SILS) procedures. Further, in some embodiments, the devices and methods disclosed herein may be used during open surgery or laparoscopic surgery. Accordingly, medical instrument 1 can be shaped, sized, and configured for insertion into the introducer, such as an endoscope.

In order for medical instrument 1 to access an internal surgical site, medical instrument 1 can be inserted into a suitable introduction sheath medical device and work in conjunction with the medical device. Sometimes, however, an outer configuration or a function of medical instrument 1 may not correspond with an inner configuration or function of the medical device. For instance, medical instrument 1 may not be compatible with a handle of the medical device, an internal lumen of the medical device, or an insertion port allowing access to the lumen of the medical device. Adaptor 3 may provide compatibility between medical instrument 1 and the medical device.

Adaptor 3 in FIG. 1 is shown mounted on medical instrument 1 so as to alter an external configuration, such as, e.g., the size, shape, or configuration of underlying medical instrument 1, and to possibly alter the function, as discussed further below. Medical instrument 1 may include any suitable instrument, for example, a surgical or minimally invasive instrument, like a snare, biopsy needle, forceps, cutting tool, clip, suction or irrigation tool, etc.

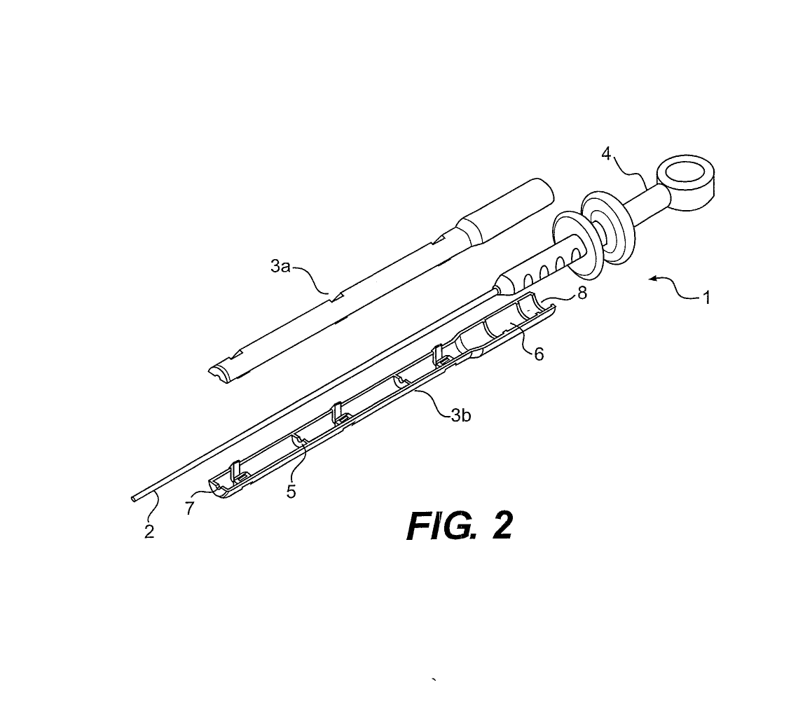

As shown in FIG. 2, adaptor 3 may include two separate sections configured to receive at least a portion of medical instrument 1 therein. Adaptor sections 3a, 3b may have an at least partially hollow center region 6 in order to contain medical instrument 1. Further, adaptor sections 3a, 3b may be similarly shaped. In this embodiment, two adaptor sections 3a, 3b are configured to sandwich medical instrument 1 between them and secure it in place. Adaptor sections 3a, 3b may include elongated halves that sandwich medical instrument 1, as is shown in FIGS. 1 and 2, or may include a plurality of elongated sections connected in a series over medical instrument 1. In the latter embodiment, each section 3a, 3b may include a plurality of elongated sections, or a plurality of sections 3a, 3b may be connected in series over a portion of the length of medical instrument 1.

Each adaptor section 3a, 3b may include one or more ribs 5 or other suitable structures configured to span a portion of center region 6, which may help to orient medical instrument 1 within adaptor sections 3a, 3b. Ribs 5 may include openings that are larger, smaller, or the same size as an outer diameter of medical instrument 1. Ribs 5 may be coated with a suitable friction-reducing material, such as TEFLON®, polyetheretherketone, polyimide, nylon, polyethylene, polytetrafluorethylene (PTFE), high-density polyethylene (HDPE), or other lubricious polymer coatings, to reduce surface friction between ribs 5 and medical instrument 1. Similarly, an outer diameter of adaptor 3 may include such a friction reducing coating. Alternatively, ribs 5 may include a coating suitable for increasing the grip and friction between ribs 5 and medical instrument 1, for instance, a polymer grip coating. Moreover, to inhibit bacterial growth in medical instrument 1 or adaptor 3, ribs 5 may be coated with an antibacterial coating. The coating may contain an inorganic antibiotic agent, disposed in a polymeric matrix, that adheres the antibiotic agent to the surface of ribs 5. Adaptor sections 3a, 3b may further include a distal opening 7 and a proximal opening 8 configured to allow portions of medical instrument 1 to protrude from a distal region of adaptor 3 and a proximal region of adaptor 3 when adaptor sections 3a, 3b are secured in place on medical instrument 1.

Adaptor sections 3a, 3b may secure in place by any means. Adaptor sections 3a, 3b may secure to each other. For instance, they may snap fit, slide together to lock, latch, or hook to one another. In one embodiment, two or more sections 3a, 3b of adaptor 3 may be connected to each other by one or more hinges and may swing open for positioning around medical instrument 1, and may swing closed and may snap or latch closed to mount on medical instrument 1. In another embodiment, adaptor sections 3a, 3b may be locked together via a locking system (not shown). In such an arrangement, one or both of adaptor sections 3a, 3b may include, for example, a slide bar or prongs thereon. The slide bar may be actuated to translate from a first position to a second position, thereby moving one or more prongs along a key slot until the one or more prongs are locked within the key slot. In a further embodiment, such a locking system need not include a slide bar. Rather, one or both of adaptor sections 3a, 3b, may be provided with one or more prongs configured to be received within a corresponding one or more key slots such that movement of one of adaptor sections 3a, 3b relative to the other of adaptor section 3b may lock the one or more prongs within the corresponding one or more key slots.

Alternatively, adaptor sections 3a, 3b may secure directly to medical instrument 1 in addition to or instead of securing to each other. For instance, adaptor sections 3a, 3b may snap fit onto medical instrument 1, slide into place around medical instrument 1, friction fit, interference fit, latched, snapped onto, or hooked to medical instrument 1. In some embodiments, adaptor sections 3a, 3b may be fit onto medical instrument 1 such that a proximal end of adaptor 3 is snugly (e.g. tightly) secured to handle 4 while a less snug (e.g. looser) connection is achieved between a distal end of adaptor 3 and a shaft 2 of medical device 1. In some embodiments, adaptor sections 3a, 3b may fit only onto handle 4 of medical instrument 1. As such, slipping may be prevented between adaptor 3 and handle 4 while avoiding kinking of the shaft 2 during assembly of adaptor 3 on medical device 1. The openings in ribs 5 may be configured to grasp medical instrument 1. For example, they may be of the same or slightly smaller diameter as medical instrument 1, or they may have a flat surface at one or more regions.

In some embodiments, a proximal portion of adaptor 3 may include a geometric structure or feature configured to interact or cooperate with a structure on medical device 1. For example, a handle of medical device 1 may include a distal disc (not shown) and adaptor 3 may include a corresponding feature for cooperating with the distal disc to minimize relative movement between the adaptor 3 and medical device 1. For example, handle 4 may include an “o” ring at the point of contact between rib 5 and medical instrument 1.

Further, although adaptor 3 is shown as including two adaptor sections 3a, 3b, adaptor 3 may include any number of sections. For instance, adaptor 3 may be formed of one continuous section. The single section could slide in place over medical instrument 1 and may lock in place through a friction fit, screw in place via a threaded region, e.g., on handle 4, or may snap fit, interference fit, or secure to medical instrument 1 by any other suitable mechanism. Alternatively, adaptor 3 may include 3 or more separate sections that fit together over medical instrument 1. Additionally, although sections 3a, 3b are shown as equal halves that join together along a seam extending in an axial direction, the sections of adaptor 3 may be unequal or nonsymmetrical and may join together along a seam that extends in an axial, non-axial, diagonal, or irregular direction. For instance, each section may have irregular edges that allow the separate sections to interlock in order to help align the sections or secure the sections to each other or to medical instrument 1.

In one embodiment, adaptor 3 may be configured to connect with handle 4 of medical instrument 1. In the exemplary depicted embodiment, adaptor 3 is elongated and shaped to fit over a portion of a shaft 2 of medical instrument 1 and a portion of a handle 4 of medical instrument 1. Adaptor 3 in this embodiment has an exterior region that is smooth and shaped to correspond to the contours of underlying medical instrument 1. Adaptor 3 may be shaped and configured to fit over any portion of medical instrument 1. For instance, adaptor 3 may encase substantially all of medical instrument 1, may be configured to fit over a portion of handle 4, a portion of shaft 2, a portion of a cord or connector connecting medical instrument 1 to, for instance, a power source, sensor, or data processor, or any other portion or combination of portions of medical instrument 1. Further, adaptor 3 may itself connect medical instrument 1 to another entity, for instance, those listed above or the medical device that medical instrument 1 is intended to be used with. Adaptor 3 may also include cutouts or openings allowing access or ventilation to underlying medical instrument 1. Additionally, multiple adaptors 3 may be arranged to fit over different portions of medical instrument 1 together. Adaptor 3 may be configured to completely surround a portion of medical instrument 1, as shown in FIG. 1, or may surround only a portion of the circumference, width, or length of medical instrument 1.

Adaptor 3 may be of any suitable configuration, size, or shape, all of which may vary depending on the type of medical instrument 1 that adaptor 3 is intended to be mounted on or the type of medical device that medical instrument 1 is intended for use with. For instance, an outer surface of adaptor 3 may follow the contours of medical instrument 1, as shown in FIGS. 1 and 2, or may have a shape or size that is different from underlying medical device 1. Further, at least one of a proximal end and a distal end of adaptor 3 may include tapered portions, or portions of reduced diameter, configured to facilitate introduction of medical instrument 1 into a lumen of the medical device. Additionally, adaptor 3 may be formed of any suitable material. For instance, adaptor 3 may be formed of plastic, e.g. any suitable polymer, a metal or metal alloy, ceramic, glass, or any other suitable material or combination thereof. Further, adaptor 3 may be reusable or disposable. Ribs 5 may comprise, for example, a soft material or a rigid material to help grasp medical instrument 1.

Further, adaptor 3 may be a separate component from both medical instrument 1 and a medical device, or adaptor 3 may be integrated into either medical instrument 1 or the medical device itself. For instance, medical instrument 1 may have portions that attach, detach, or adjust to work with different medical devices or different adaptors on different medical devices. These portions may be configured for storing on or in medical instrument 1. Further, a medical device may include or may store different adaptors configured to accept different medical instruments 1.

Adaptor 3 may allow medical instrument 1 to be used interchangeably between multiple medical devices. For instance, adaptor 3 may allow medical instrument 1 to be used with multiple types of medical devices, such as endoscopy systems, laparoscopes, resectoscopes or other types of scopes, guide tubes, access catheters, trocars, robotic systems, or any other suitable medical devices. Adaptor 3 may allow medical instrument 1 to function and be compatible for use with different models of similarly functioning devices, different lines of commercial systems within the same brand, different brands of the same type of medical device, or across different types of medical devices. For instance, adaptor 3 may allow a single medical instrument 1 to be used interchangeably with the Boston Scientific Direct Drive Endoscopic System™ (DDES), the Transenterix Spider®, the Storz Anubis®, or the Covidien SILS™, for example. Additionally, adaptor 3 may allow medical instrument 1 to be used with any type of scope, or to switch between use with a scope and a trocar, for example.

Adaptor 3 may provide basic structural adjustments to allow compatibility with different medical devices. For instance, adaptor 3 may increase or decrease length, width, cross-section, opening sizes, etc. to medical instrument 1, or may provide structural elements, like ridges, projections, inserts, or openings that may be required of an instrument for use with certain medical devices. For example, adaptor 3 may include portions capable of, e.g., telescoping, twisting, or snap-fitting to adjust. For example, such telescoping portions of adaptor 3 may assist in adjusting a length of the underlying medical instrument 1. Further, depending on the function of medical instrument 1 or a medical device for use with medical instrument 1, adaptor 3 may securely attach to medical instrument 1 so as to restrict movement, or may be moveable when secured to medical instrument 1 to allow, for instance, rotational, sliding, or other types of movement. In some embodiments, the same adaptor 3 may be capable of both moving and nonmoving attachment. In addition, some medical devices may only be compatible with specific materials, in which case, adaptor 3 may be made of the correct corresponding material.

In addition to requiring different sized or shaped medical instruments 1, different types of medical procedures, medical devices, models, or brands, may require different functionalities. Thus, the functionalities needed of medical instrument 1 may vary greatly. In one example, a physician may require different functionalities when using different types of scopes or other introduction sheaths. Alternatively, physicians may require different functionalities of the same scopes depending on the procedure to be performed. Rather than maintaining separate inventories of medical instrument 1 for each type of scope or each surgical procedure, adaptor 3 may allow medical instrument 1 to be used with any type of scope, regardless of the different functions.

In another example, different models of the same type of medical device may include different features and functionalities that older or lower-end models lack. Further, if the features are the same, the features' function or the dimensions of the medical device or medical instrument 1 may change over time. Physicians wishing to upgrade models may not want to repurchase a new set of medical instruments 1 to use with the newer model. Additionally, a hospital might simultaneously have both older and newer or low-end and high-end models, and may wish to keep one inventory of medical instruments for use with all models rather than stockpile separate medical instruments for each. Certain exemplary embodiments of adaptor 3 may alter the functionality of underlying medical instrument 1. For instance, adaptor 3 may allow medical instrument 1 to interface with different medical devices, e.g., surgical systems, and guide, slide, rotate, retain a position, articulate, or otherwise function within different systems. Adaptor 3 may be flexible, rigid, or may have variable flexibility, or may have various flexible and rigid portions along its length. Further, adaptor 3 may provide additional information. For example, adaptor 3 may include one or more markings visible to a user to indicate distance, rotational angles, displacement, etc.

For instance, when adaptor 3 is used to affect the functionality of medical instrument 1, adaptor 3 may alter a function of medical instrument 1 that is already present in order to align the functionality with that of a medical device. For example, adaptor 3 could alter or control the ease or resistance of movement, e.g., braking, locking, rotating, sliding, articulating, actuating, etc., of medical instrument 1 to match the instrument movement-to-input signal ratio that the medical device was designed to pair with. Additionally, adaptor 3 may allow a user to make medical instrument 1 more ergonomically comfortable, for instance to switch the orientation of medical instrument 1, for instance, helping to configure a right-handed instrument for a left-handed user, or vice versa.

Adaptor 3 may also add a function to medical instrument 1 that a medical device is designed to have or is capable of handling, but that the instrument itself lacks. For instance, center region 6 of adaptor 3 or an interior or exterior portion of adaptor 3 may include controls or connectors that integrate with medical instrument 1 to offer additional functionalities. For instance, adaptor 3 may include a brake system that operably couples to medical instrument 1 when adaptor 3 is mounted on instrument 1, and may act, for instance, on shaft 2 or handle 4, to permit locking or positioning of medical instrument 1. In other embodiments, adaptor 3 may add such functionalities as allowing medical instrument 1 to rotate or retract, or to provide suction or irrigation, for example. Further, adaptor 3 may provide suction, irrigation or other functionalities, and may be configured to operably couple with a fluid source, a power source, or other equipment external to medical instrument 1 or the medical device. Adaptor 3 may include one or more additional lumens. For instance, adaptor 3 may provide additional lumens that may allow additional surgical instruments to be delivered through adaptor 3. Adaptor 3 may also allow insertion of imaging systems, such as a fiber optic or solid state imaging chip, for example, to a distal region of medical instrument 1. Additionally, adaptor 3 may increase a stiffness, leverage, reach, and or torque transferring ability of the underlying medical instrument 1. In other embodiments, adaptor 3 may be configured to operably connect with an external controller, memory, or storage device, for instance. This connection may be wireless or physical. Adaptor 3 may include one or more sensors to receive or transmit data from medical instrument 1 or the medical device, or to take a reading from the area into which medical instrument 1 is inserted. Such a sensor could, for example, provide feedback regarding the functioning status of adaptor 3, medical instrument 1, or the medical device, or it could provide information regarding a patient or a work site, such as temperature, pressure, a constituent concentration, etc. Adaptor 3 may also make medical instrument 1 waterproof or air-tight to meet requirements for use with different medical devices.

Adaptor 3 may further include control mechanisms on an outer region configured to allow a user to actuate or otherwise control certain functions of medical instrument 1. Such control mechanisms could include, e.g., a button, lever, switch, knob, screw wheel, thumb wheel, ratchet, gear, crank, spring etc., or any combination thereof.

Adaptor 3 may also be capable of overriding or removing a function from medical instrument 1. For instance, medical instrument 1 may be intended for use with a newer or higher-end model capable of a function that an older or lower-end model does not offer. In this case, to allow use with the older or lower-end model, adaptor 3 may cancel out this function of medical instrument 1. For example, newer medical instrument 1 may be capable of rotational movement, but older medical devices may not be configured to allow medical instrument 1 to rotate. Adaptor 3 may be configured to disable the rotational function. In another example, medical instrument 1 may be capable of increased articulation above what a medical device is capable of allowing for. For instance, a newer medical instrument 1 may be configured to allow for vertical, horizontal, and diagonal articulation of a distal end of instrument 1, but an older device may only be capable of allowing vertical and horizontal movement. In this scenario, adaptor 3 may be configured to cancel out the diagonal movement. This may be accomplished, for instance, by preventing such movement in shaft 2, or changing the configuration of handle 4, for instance. These functions are exemplary, and adaptor 3 may be configured to cancel out one or more functions of medical instrument 1 in order to make medical instrument 1 compatible with a medical device used for introducing medical instrument 1 into a patient.

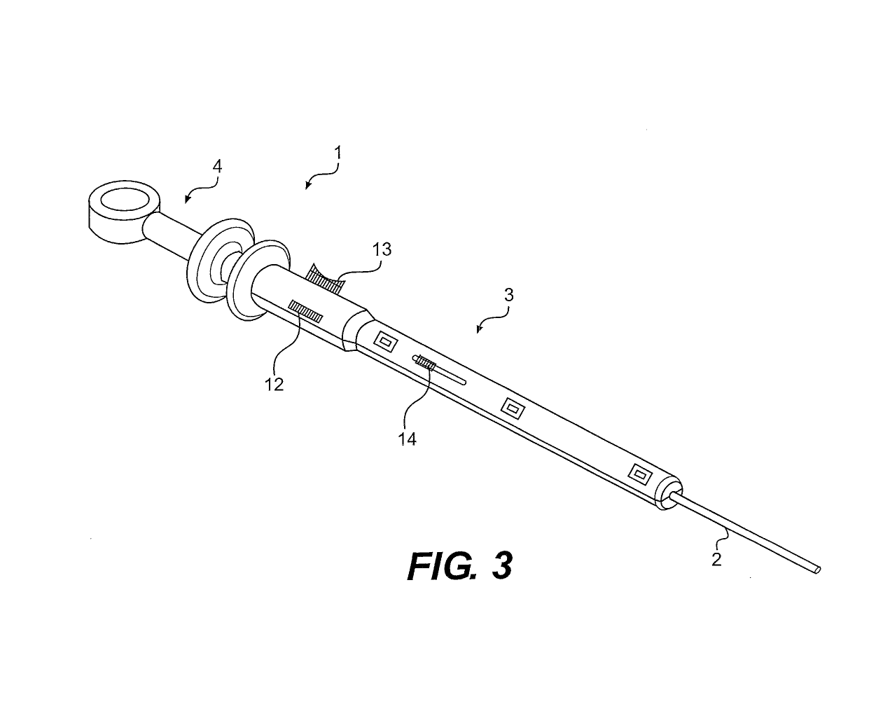

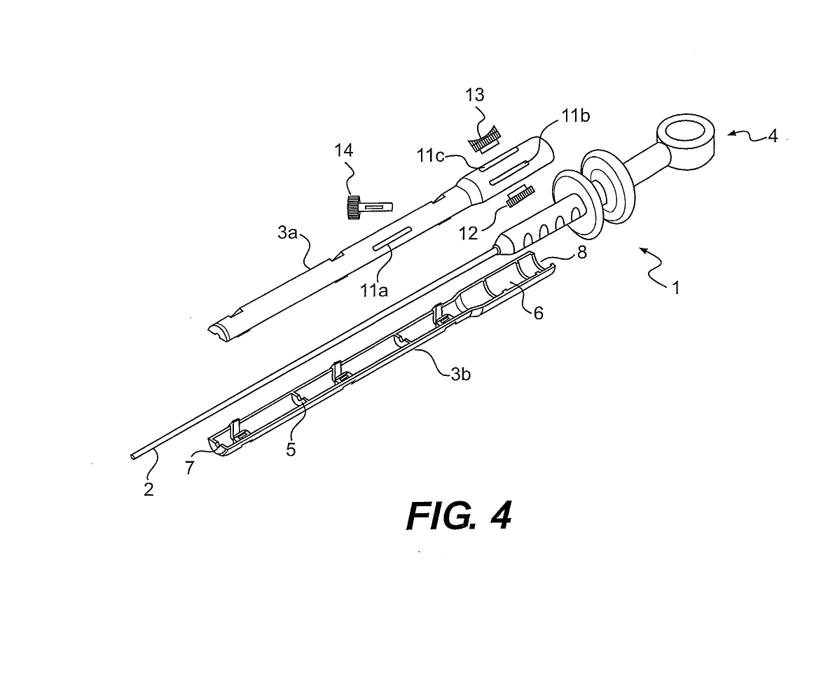

In other exemplary embodiments, adaptor 3 may be modular to help accommodate different functionalities. As is shown in FIGS. 3 and 4, sections 3a, 3b may be configured for use with multiple different elements that may be interchanged for use with adaptor 3 to accommodate the different functions or capabilities of medical instrument 1 or a medical device for use with medical instrument 1. Adaptor 3, for instance, may include a kit comprising multiple elements, e.g., control elements, such as actuators (lever 14, buttons (not shown), switches 13, etc.), non-control plug elements 12, or any other suitable elements that a user can choose between depending on the function, type of medical instrument 1, type of medical device, or intended use of medical instrument 1. In one embodiment, adaptor 3 may include a plurality of openings 11a, 11b, 11c. Though FIGS. 3 and 4 show three rectangular openings, any number, shape, or sized openings may be included in adaptor 3. Each opening 11a, 11b, 11c may be configured to receive either plug element 12 to at least partially cover the opening, or alternatively, an actuator element 13, 14, connector (not shown), or some other control element. For example, a medical instrument 1 may lack a function for use with a medical device that adaptor 3 can provide. Accordingly, medical instrument 1 may lack a suitable control mechanism to activate the new function provided by adaptor 3. In order to actuate this new function, adaptor 3 may be configured to provide the control element.

Openings 11a, 11b, 11c may be configured to receive different elements, and a user may arrange the elements in these openings to configure adaptor 3 to include a control element appropriate for the added function. This may involve inserting plug elements 12 configured to cover an opening into all but one of openings 11a, 11b, 11c and inserting a suitable control element 13 into one opening to accommodate the added function. In another instance, medical instrument 1 may lack two or more functions that adaptor 3 is capable of providing. In this scenario, a user may configure adaptor 3 to include two or more control elements 13, 14 for actuating the added features. Accordingly, plug elements 12 may be inserted into all openings 11a, 11b, 11c except those in which control elements are needed to provide actuation for the number of functions needed. In other embodiments, one actuation element may be capable of actuating more than one function, or more than one control element may be used to actuate the same function. In these situations, a user can adjust the number of plug elements 12 and the number of control elements 13, 14 accordingly inserted into openings 11a, 11b, 11c. Further examples of such modularity are described in U.S. Provisional Application No. 61/593,209, which is incorporated herein in its entirety by reference.

Any number of adaptors 3 may be provided in a kit, or group of adaptors 3. In such an exemplary embodiment, each adaptor 3 may be labeled or otherwise imprinted with information thereon. Indeed, each adaptor 3 may be imprinted with a proprietary logo, instructions, or indications for assisting a user. For instance, an adaptor 3 may be imprinted with a brand name or device name so as to convey to the user which medical devices 1 may appropriately be used therewith.

The many features and advantages of the present disclosure are apparent from the detailed specification, and thus, it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described, and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. It is important, therefore, to recognize that the claims should be regarded as including such equivalent constructions insofar as they do not depart from the spirit and scope of the present disclosure.

What is claimed is:

1. An adaptor for a medical instrument configured to be inserted into a body of a patient, comprising:
a housing defining an internal recess configured to receive a portion of the medical instrument, wherein the internal recess includes geometric configurations configured to secure the housing to the medical instrument, wherein the outer configuration of the adaptor is compatible with a medical device.

2. The adaptor of claim 1, wherein the housing includes a plurality of sections.

3. The adaptor of claim 1, wherein the outer configuration of the adaptor differs from the outer configuration of the medical instrument in at least one of a shape, a size, and a function.

4. The adaptor of claim 1, wherein a proximal portion of the housing is configured to receive a handle portion of the medical instrument.

5. The adaptor of claim 1, wherein the medical instrument includes an elongate shaft having a proximal and a distal end, a handle disposed at the proximal end, and an end-effector extending from the distal end.

6. The adaptor of claim 1, wherein the internal recess includes at least one supporting structure for supporting a portion of the medical instrument therein.

7. The adaptor of claim 1, wherein the at least one supporting structure includes a plurality of circumferential ribs.

8. The adaptor of claim 1, wherein the housing includes a proximal portion having a first diameter and a distal portion having a second diameter different from the first diameter.

9. The adaptor of claim 2, wherein the plurality of sections are configured to be secured together by one of an interference fit and a friction fit.

10. The adaptor of claim 1, wherein the housing is directly connected to the medical instrument.

11. The adaptor of claim 1, wherein the adaptor includes at least one control element configured to control at least one feature of the medical instrument.

12. An medical system, comprising:
an introduction sheath having a proximal end, a distal end, and a lumen extending therebetween, wherein the proximal end includes a handle having a port in communication with the lumen;
a medical instrument having an outer configuration incompatible with the port; and
an adaptor comprising:
a housing defining an internal recess configured to receive a portion of the medical instrument, wherein the internal recess is configured to generally correspond to an outer configuration of the portion of the medical instrument; and
a securing mechanism for securing the housing about the medical instrument,
wherein the adaptor includes an outer configuration compatible with the port.

13. The medical system of claim 12, wherein the introduction sheath includes an endoscope having a plurality of lumens.

14. The medical system of claim 12, wherein the housing includes a plurality of sections.

15. The medical system of claim 14, wherein the plurality of sections are secured together by at least one of an interference fit, a snap fit, and a friction fit.

16. The medidal system of claim 12, wherein the outer configuration of the adaptor differs from the outer configuration of the medical instrument.

17. The medical system of claim 12, wherein a proximal portion of the housing is configured to receive a handle portion of the medical instrument.

18. The medical system of claim 12, wherein the adaptor is configured for use with different types of medical instruments and different types of introduction sheaths.

19. A medical method comprising:
advancing an introduction sheath to a target location within a body of a patient, wherein the introduction sheath includes a working channel;
securing an adaptor to an exterior of a medical instrument, wherein the adaptor comprises:
a housing defining an internal recess configured to receive a portion of the medical instrument, wherein the internal recess is configured to generally correspond to an outer configuration of the portion of the medical instrument, wherein the adaptor includes an outer configuration compatible with the working channel; and
advancing the medical instrument and the adaptor into the working channel of the introduction sheath.

20. The medical method of claim 19, wherein the housing includes a plurality of sections.
